(19) United States
(12) Patent Application Publication
(10) Pub. No.: US 2016/0174897 A1
(43) Pub. Date: Jun. 23, 2016

(54) Sensor Embedded Wearable Technology Glove

(71) Applicant: Caterpillar Inc., Peoria, IL (US)

(72) Inventor: John Sherman, Peoria, IL (US)

(73) Assignee: CATERPILLAR INC., Peoria, IL (US)

(21) Appl. No.: 14/578,796

(22) Filed: Dec. 22, 2014

(51) Int. Cl. A61B 5/00; A61B 5/01; A61B 5/11; A61B 19/00

(52) U.S. Cl. CPC A61B 5/6806; A61B 19/54; A61B 5/0059; A61B 5/01; A61B 5/7282; A61B 5/1127; A61B 2019/5445; A61B 2562/164; A61B 2503/20; A61B 2560/0242

(57) ABSTRACT

An apparatus for monitoring a condition of a human hand, relative to a reference environment, is disclosed. The apparatus includes at least one fiducial marker. The fiducial marker is in the field of view of an imaging system and the imaging system is designed for monitoring the human hand via the fiducial marker within the reference environment at a discrete point in time. The apparatus further includes at least one sensor capable of detecting an impact event associated with the condition of the human hand. The sensor may generate a signal indicative of the condition of the human hand at the discrete point in time and within the reference environment.

### TECHNICAL FIELD

The present disclosure generally relates to systems and methods for monitoring movement in a working environment for ergonomic research, and, more particularly, to apparatus and methods for monitoring conditions of a human hand within a working environment.

### BACKGROUND

Within a modern working environment, there is a desire, from both the employee and employer, to optimize the workplace (e.g., a factory) for a variety of reasons, such as performance operation and safety. As such, a variety of systems and methods have been developed to detect position data representative of the movement, kinematics, and position of the extremities of operating workers within the workplace. Using such detected position data, a variety of workplace conditions can be evaluated, such as, but not limited to, ergonomic conditions of the operator and/or workplace, optimization of spatial relationships between operators and machinery, safety of workers and machines within the workplace, and the like. However, there is a limitless amount of workplace condition analysis that can be performed using such detected position data.

Current ergonomic analysis systems may employ machine vision sensors to monitor and track data associated with operators' gross position and/or configuration of the extremities of an operator's body (e.g., monitoring position and movement of an operator's arms and legs within the workplace). Such systems may generate rough estimates of human body kinematic positions and movement information by capturing images of the operator's body using an imaging system, such as one or more cameras. Using the information gathered, models may be generated using a computer showing motion of arms and legs of the user.

While position data regarding the arms and legs of the operator is important and useful in ergonomic analysis, information of higher resolution is always more desirable. In fact, one of the most desired areas for continued analysis upon the human body is the human hand. However, the imaging systems described often do not have a high enough resolution to properly analyze the motion and distinct characteristics of hand movement within such imaging systems.

In the medical field, research has been done to examine certain characteristics of the human hand using wearable technology, like a glove. For example, systems have been developed that monitor motion of arthritis patients using a glove that can detect and provide information to electronically model joint movement of the fingers and thumbs. Further, some wearable technology exists that employs sensors to detect motion and subsequently output signals indicative of data to be input to another system. For example, wearable gloves exist that can be used to generate input data, to a machine, based on specific hand motions made by the hand and detected by sensors associated with the glove, as described in U.S. Pat. No. 4,414,537 (“Digital Data Entry Glove Interface Device”).

However, such systems and methods do not provide high fidelity ergonomics data associated with the hand, within the reference environment of the workplace. Therefore, a need exists for apparatus and methods for detecting motion of a human hand, within a workplace environment, in conjunction with a larger imaging system for determining ergonomic impact of workplace conditions.

### SUMMARY

In accordance with one aspect of the disclosure, an apparatus for monitoring a condition of a human hand, relative to a reference environment, is disclosed. The apparatus may include at least one fiducial marker. The fiducial marker may be in the field of view of an imaging system and the imaging system is designed for monitoring the human hand via the fiducial marker within the reference environment at a discrete point in time. The apparatus may further include at least one sensor capable of detecting an impact event associated with the condition of the human hand. The sensor may generate a signal indicative of the condition of the human hand at the discrete point in time and within the reference environment. In some examples, the condition of the human hand may include, but is not limited to including the condition associated with the human hand includes at least one of a hand location, a hand orientation, a joint position of a joint of the human hand, a movement of the human hand, a repetition of hand movement, a pressure on the human hand, or a vibration of the human hand.

In accordance with another aspect of the disclosure, a method for monitoring a condition of a human hand, relative to a reference environment, is disclosed. The method may include detecting the human hand using an imaging system at a discrete point in time. The imaging system may detect the human hand by detecting at least one fiducial marker associated with the human hand. The method may further include detecting an impact event associated with the condition of the human hand using at least one sensor and generating a signal indicative of the condition of the human hand the discrete point in time and within the reference environment. In some examples, the method may further include providing the signal indicative of the condition of the human hand to a computer, analyzing the signal indicative of the human hand for ergonomic monitoring of the human hand, and generating ergonomic data associated with the human hand.

In accordance with yet another aspect of the disclosure, a glove, which is wearable on a human hand and configured for monitoring a condition of the human hand relative to a reference environment, is disclosed. The glove may include a fabric, the fabric being movable with the human hand when the glove is worn by the human hand. The glove may further include at least one fiducial marker, the fiducial marker being in the field of view of an imaging system. The imaging system may be designed for monitoring the human hand via the fiducial marker within the reference environment at a discrete point in time. The glove may further include a plurality of sensors operatively associated with the fabric, the plurality of sensors including at least one impact sensor capable of detecting an impact event associated with the condition of the human hand at the at least one discrete point in time and within the reference environment. In some examples, the plurality of sensors may include fabric-embedded sensors.

These and other aspects and features of the present disclosure will be better understood when read in conjunction with the accompanying drawings.

### BRIEF DESCRIPTION OF THE DRAWINGS

FIG. 1 is a schematic view of a sensor embedded glove that shows the glove, as worn by a human hand, from the top side of the hand, in accordance with the present disclosure.

FIG. 2 is a schematic view of the sensor embedded glove of FIG. 1 from the palm side of the hand, in accordance with the present disclosure.

FIG. 3 is a schematic view of the sensor embedded glove of FIGS. 1 and 2, from a side perspective while posed, in accordance with the present disclosure.

FIG. 4 is a perspective view of a working environment in which the sensor embedded glove of FIGS. 1-3 may monitor conditions associated with the human hand, in accordance with the present disclosure.

FIG. 5 is a schematic block diagram showing schematic arrangement of the sensor embedded glove of FIGS. 1-3 and its associated elements in operative association with a computer and an imaging system, in accordance with the present disclosure.

FIG. 6 is a flow chart for describing a method for monitoring a condition of a human hand, relative to a reference environment, in accordance with the present disclosure.

While the following detailed description will be given with respect to certain illustrative embodiments, it should be understood that the drawings are not necessarily to scale and the disclosed embodiments are sometimes illustrated diagrammatically and in partial views. In addition, in certain instances, details which are not necessary for an understanding of the disclosed subject matter or which render other details too difficult to perceive may have been omitted. It should therefore be understood that this disclosure is not limited to the particular embodiments disclosed and illustrated herein, but rather to a fair reading of the entire disclosure and claims, as well as any equivalents thereto.

### DETAILED DESCRIPTION

The present disclosure provides systems, methods, and apparatus for monitoring conditions of human hands, relative to a reference environment, which may be used to generate data for ergonomics research and analysis. The hand condition data may be generated using one or more sensor embedded gloves which may communicate said data to a computing device for processing the information. The data generated from the glove and the associated sensors may be used, but is not limited to being used, for the purposes of generating task related ergonomic impact data, environmental health data, environmental safety data, ergonomic automation data, or any other data which may be derived from sensed impact on parts of the human hand.

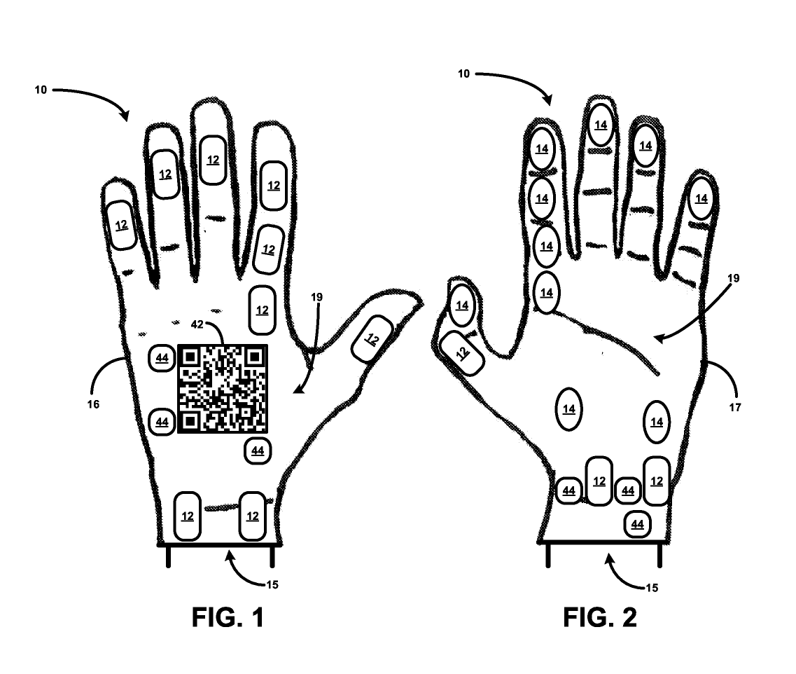

Turning now to the drawings, FIGS. 1 and 2 show a glove 10 which may include a variety of impact sensors (e.g., extension sensors 12 and pressure sensors 14). FIG. 1 shows a view of the glove 10, when worn on a human hand 15, from a top side 16 of the hand 15; whereas, FIG. 2 shows a view of the glove 10, when worn on the human hand 15, from a palm side 17 of the hand 15. The glove 10 may be formed from a fabric 19, on which the extension sensors 12 and the pressure sensors 14 may be affixed. Additionally or alternatively, the extension sensors 12 and the pressure sensors 14 may be embedded within the fabric 19. When the glove 10 is worn, the fabric 19, generally, moves with the hand 15 and specific portions of the hand (e.g., fingers, thumb, joint, etc.).

The extension sensors 12 and pressure sensors 14 may be used to generate signals indicative of any type of movement that may be associated with a condition of the human hand 15. Such movements may include any motions, vibrations, extensions, flexion, pressures, and the like. Conditions which may be detected based on said movements may include, but are certainly not limited to, hand 15 locations, hand 15 orientations, associated joint positions, hand 15 movement, repetitions of specific hand 15 movements, pressures on the hand 15, pressures on specific parts and/or regions of the hand 15, extensions of fingers and/or joints of the hand 15, extensions of a thumb and/or joints associated with the hand 15, general movement and/or flexion about a wrist associated with the hand 15, and the like.

As such, specific members of the groups of extension sensors 12 and pressure sensors 14 may be placed in locations associated with elements of the hand 15 (e.g., fingers, joints, knuckles, palms, wrists, etc.) to specifically measure data with respect to the specific location on the hand. For example, some pressure sensors 14 may be specifically located on the palm side 17 of the hand 15 to measure pressure forces on the palm side 17 of the hand. Even more specifically, certain locations of pressure sensors 14 on the palm side 17 may be useful for measuring pressures associated with finger depression on, for example, workplace materials and workspaces. In another example, the extension sensors 12 may be disposed on the top side 16 of the hand to measure extension of certain areas of the hand 15. For example, the extension sensors 12 may be disposed relative to individual joints and/or knuckles of the hand 15 to measure movement associated with said individual joints and/or knuckles of the hand 15. While the depiction of the glove 10 in the drawings of FIGS. 1 and 2 show certain example locations of the extension sensors 12 and pressure sensors 14, these positions are merely exemplary and certainly not limiting. Any combination of position(s) of one or more of the extension sensors 12 and/or the pressure sensors 14 may be used to achieve the objective of generating information associated with a condition of the hand 15.

As mentioned above, any of the extension sensors 12 and pressure sensors 14 may be embedded within the fabric 19 of the glove 10. Any of the extension sensors 12 and/or pressure sensors 14 may be embodied by one or more of, for example, a soft textile sensor, a conductive elastic yarn, an elastomeric polymer, an elastic conductive ribbon, and the like. A soft textile sensor refers to sensors used, generally, in wearable vital sign monitoring to sense electrical activity of the body, such as mechanical movements like extension and pressure. A conductive elastic yarn refers to any family of conductive elastic yarns which are used to weave or knit conductive and/or optical fabric structures. Elastomeric polymers are conductive polymers that exhibit changes in electrical conductivity as the material is stretched. Such elastomeric polymers may be nano-composite polymers and may have variable resistance properties. Structures built from such polymers may behave as strain gauges, switches, and/or sensors. Further, elastic conductive ribbons refer to ribbons that attach to electronic connectors to provide a fabric-like, motion-absorbing, wiring harness useful for wearable sensing technology applications.

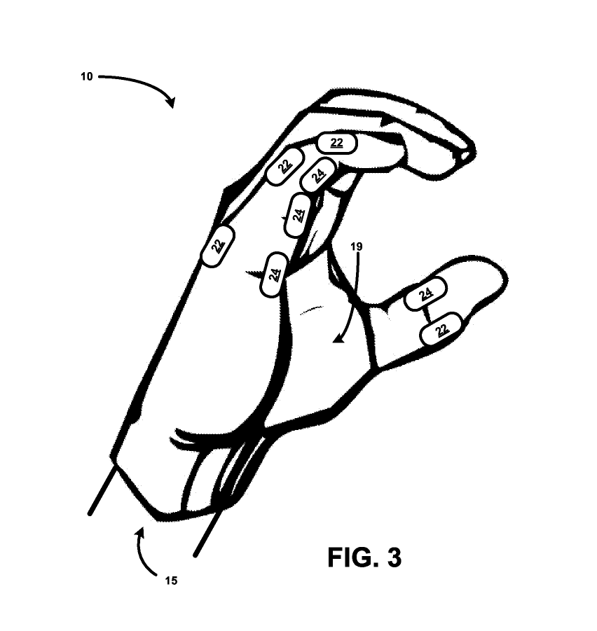

If the glove 10 involves impact sensors that are embedded within the fabric 19, as detailed above, portions of the fabric 19 may be subdivided into sensing zones for measuring impact events, such as extension and compression events. As shown in FIG. 3, the glove 10 has subdivided impact regions in the form of extension zones 22 and compression zones 24, which are formed from the aforementioned fabric-embedded sensor technologies. As the hand 15 is shown posing in a manner, such posing may cause one or more of the extension zones 22 and the compression zones 24 to detect the motion caused by the posing of the hand. Similar to the extension sensors 12 of FIGS. 1 and 2, each of the extension zones 22 are capable of providing a signal indicative of an extension event at its respective location on the fabric 19 of the glove 10.

Likewise, and similarly to the pressure sensors 14 shown in FIGS. 1 and 2, the compression zones 24 may be capable of providing a signal indicative of a compression event associated with the hand 15 at that zone of the fabric 19. Further, in the example embodiment of FIG. 3, the extension zones 22 may be located on an outer portion of the fabric 19, aligned with the top side of the hand 16. In some such examples, the extension zones 22 may be aligned with the exterior of a joint of the hand 15. Also in the example embodiment of FIG. 3, the compression zones 24 may be located on the fabric 19 at the portion aligned with the palm side 17 of the hand 15. In some such examples, the compression zones 24 may be aligned with the interior of a joint of the human hand 15. However, the alignments of the extension zones 22 and compression zones 24 shown in FIG. 3 and described herein are merely exemplary. Any number, combination, and/or alignment of extension zones 22 and/or compression zones embedded within the fabric 19 may be used, so long as the extension zones 22 and/or compression zones 24 are capable of providing information associated with a condition of the hand 15.

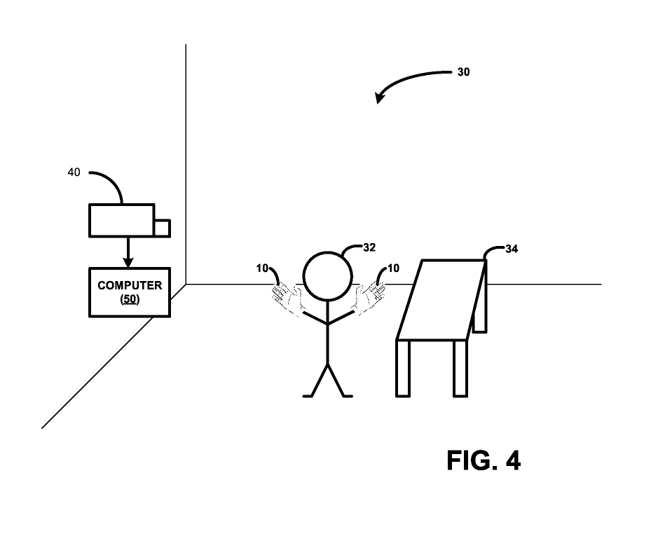

Turning now to FIG. 4, and with continued reference to FIGS. 1-3, a working environment 30 is shown. The working environment 30 may be any workplace in which an operator 32 performs tasks which may be observed for ergonomic research and analysis. The operator 32 is shown at a workstation 34; however, the working environment 30 does not necessarily need to include a workstation 34. Further, images indicative of data representative of the movement, kinematics, and position of the operator 32 and his/her extremities may be monitored by an imaging system 40. The imaging system 40 may be any imaging system capable of visually recognizing the operator 32 and/or any accessories associated with the operator (e.g., the glove 10) within the working environment 30. The imaging system 40 may include one or more cameras for capturing an image and/or any combination of sensors or detectors which may visually detect indicators from the operator 32 and/or any accessories associated with the operator 32.

In FIG. 4, the operator 32 is shown wearing the glove 10 within the working environment 30. The imaging system 40 may detect the glove 10 via one or more fiducial markers, such as the passive fiducial marker 42 and the active fiducial markers 44. The term “fiducial marker” refers to any object used in the field of view (e.g., the working environment 30) of the imaging system 40 for use as a point of reference and measure for the glove 10. The passive fiducial marker 42 may be an image imprinted on the glove 10 having a unique code embedded within that can be recognized by the imaging system 40 as unique to the glove 10. For example, the passive fiducial marker 42 may be a Quick Response (“QR”) code, as shown in FIG. 1; however, the passive fiducial marker is certainly not limited to being a QR code. Additionally or alternatively, the glove 10 may employ one or more active fiducial markers 44 for as a reference marker for the glove 10 and/or specific areas of the glove 10 to be identified by the imaging system 40. An example of an active fiducial marker 44 for use in such a glove may be a light emitting diode (LED) affixed to or embedded within the glove 10. Such LED based active fiducial markers 44 may emit light at a specific frequency, such that the imaging system 40 recognizes a unique identifier of the glove 10. Additionally or alternatively, active fiducial markers 44 may emit light in pulses in a specific pattern or pulse frequency indicative of the hand 15 and/or associated, specific portions of the hand 15.

Information provided by the imaging system 40 and the sensor embedded glove 10 may be correlated by taking readings from the sensor at one or more discrete points in time when identified by the imaging system 40, via a fiducial marker 42, 44. The sensors 12, 14, 22, 24 may provide signals indicative of a condition of the hand 15 by sensing an impact event at the one or more discrete points in time. The information provided may be analyzed, monitored, collected, stored, and/or otherwise used in relation to ergonomics associated with the working environment 30 and the operator 32.

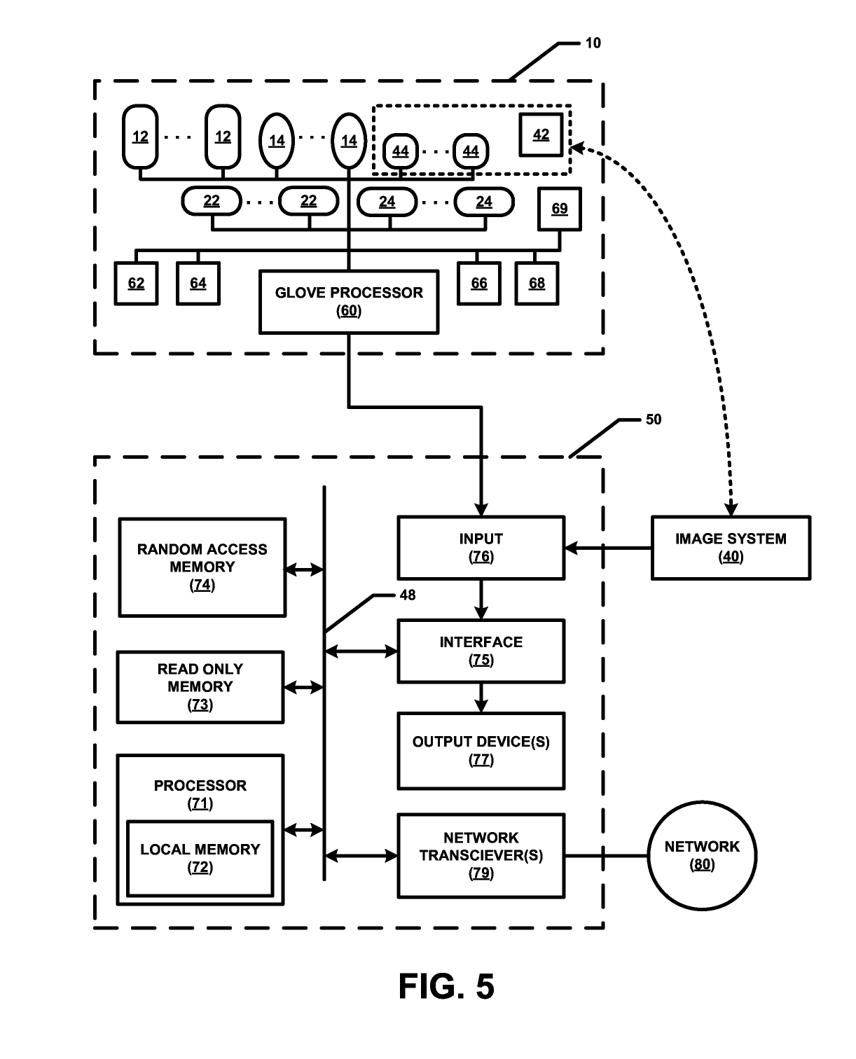

The imaging system 40 and the glove 10 may be operatively associated with a computer 50 for receiving and processing the data provided by both the imaging system 40 and the glove 10. A schematic representation of interaction amongst the glove 10, imaging system 40, and computer 50 is shown in FIG. 5. Beginning with the glove 10, the schematic representation shows the plurality of extension sensors 12, plurality of pressure sensors 14, plurality of extension zones 22, plurality of compression zones 24, and plurality of active fiducial markers 44 connected to a glove processor 60. The glove processor 60 may be any processor associated with the glove 10 which may receive information from the sensors and provide them to the computer 50. In some examples, the glove may further include one or more of a temperature sensor 62, a vibration sensor 64, a gesture sensor 66, a galvanic skin response sensor 68, or any other sensor(s) 69 which may provide information associated with a condition of the hand 15 for ergonomic analysis and all of which may be also connected to the glove processor 60. The imaging system 40 is shown visually associated with the glove 10, as the field of view of the imaging system 40 may include the passive fiducial marker 42 and the active fiducial markers 44.

The computer 50 is shown as a block diagram of a computer capable of executing instructions for receiving information from the glove 10 and the imaging system 40 and for analyzing the received information The computer 50 may be, for example, a server, a personal computer, or any other type of computing device. The computer 50 of the instant example includes a processor 71. For example, the processor 71 may be implemented by one or more microprocessors or controllers from any desired family or manufacturer.

The processor 71 includes a local memory 72 and is in communication with a main memory including a read only memory 73 and a random access memory 74 via a bus 78. The random access memory 74 may be implemented by Synchronous Dynamic Random Access Memory (SDRAM), Dynamic Random Access Memory (DRAM), RAMBUS Dynamic Random Access Memory (RDRAM) and/or any other type of random access memory device. The read only memory 73 may be implemented by a hard drive, flash memory and/or any other desired type of memory device.

The computer 50 may also include an interface circuit 75. The interface circuit 75 may be implemented by any type of interface standard, such as, for example, an Ethernet interface, a universal serial bus (USB), and/or a PCI express interface. One or more input devices 76 are connected to the interface circuit 75. The input device(s) 76 permit a user to enter data and commands into the processor 71. The input device(s) 76 can be implemented by, for example, a keyboard, a mouse, a touchscreen, a track-pad, a trackball, and/or a voice recognition system. For example, the input device(s) 76 may include any wired or wireless device for connecting the computer 50 to the imaging system 40 and the glove 10 to receive data. The input 76 may be implemented using any wired connection or wireless communication method to receive information from the imaging system and the glove 10.

One or more output devices 77 may be connected to the interface circuit 75. The output devices 77 can be implemented by, for example, display devices for associated data (e.g., a liquid crystal display, a cathode ray tube display (CRT), etc.). Further, the computer 50 may include one or more network transceivers 79 for connecting to a network 80, such as the Internet, a WLAN, a LAN, a personal network, or any other network for connecting the computer 77 to one or more other computers or network capable devices. Further, the computer 50 may be implemented using more than one computing device for analyzing data received from the glove 10 and imaging system 40.

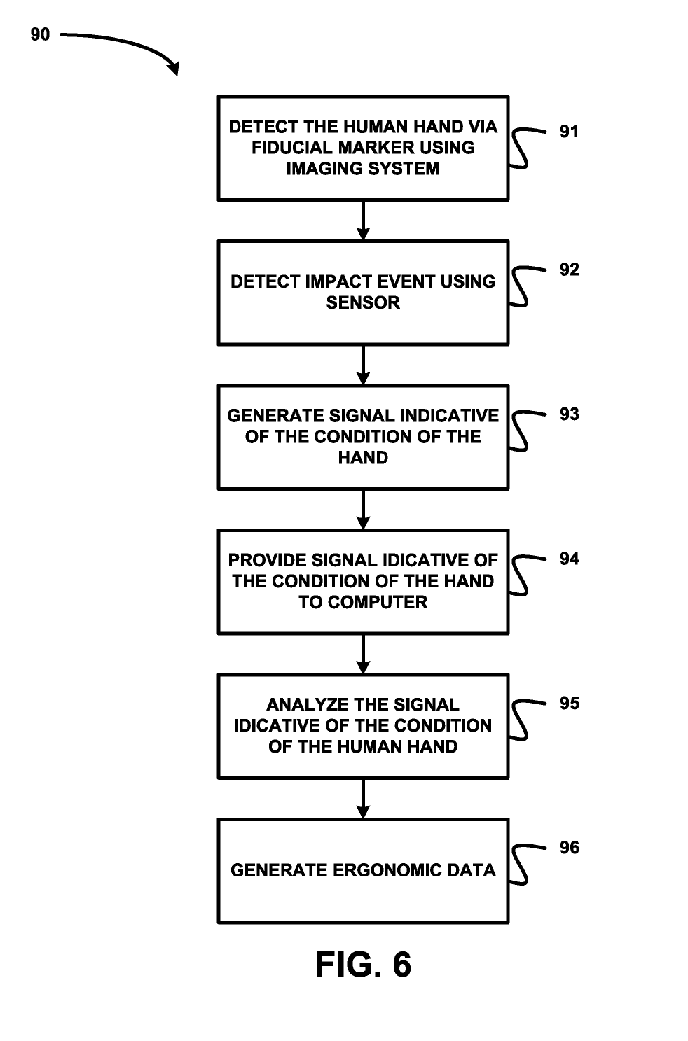

As mentioned above the computer 50 may be used to execute machine readable instructions. For example, the computer 50 may execute machine readable instructions to perform the methods shown in the block diagrams of FIG. 6 and described in more detail below. In such examples, the machine readable instructions comprise a program for execution by a processor such as the processor 71 shown in the example computer 50. The program may be embodied in software stored on a tangible computer readable medium such as a CD-ROM, a floppy disk, a hard drive, a digital versatile disk (DVD), a Blu-ray disk, or a memory associated with the processor 71, but the entire program and/or parts thereof could alternatively be executed by a device other than the processor 71 and/or embodied in firmware or dedicated hardware. Further, although the example programs are described with reference to the flowcharts illustrated in FIG. 6, many other methods of implementing embodiments of the present disclosure may alternatively be used. For example, the order of execution of the blocks may be changed, and/or some of the blocks described may be changed, eliminated, or combined.

### INDUSTRIAL APPLICABILITY

The present disclosure generally relates to systems and methods for monitoring movement in a working environment for ergonomics, and, more particularly, to apparatus and methods for monitoring conditions of a human hand within a working environment. The present disclosure provides systems, methods, and apparatus for monitoring conditions of human hands, relative to a reference environment, which may be used to generate data for ergonomics research and analysis. The hand condition data may be generated using one or more sensor embedded gloves which may communicate said data to a computing device for processing the information. The data generated from the glove and the associated sensors may be used, but is not limited to being used, for the purposes of generating task related ergonomic impact data, environmental health data, environmental safety data, ergonomic automation data, or any other data which may be derived from sensed impact on parts of the human hand.

A method 90 for monitoring such conditions of a human hand, relative to a reference environment (e.g., the working environment 30) is shown in the flowchart of FIG. 6. The method 90 begins by using the imaging system 40 to detect the human hand 15 (via the glove 10) at a point in time (block 91). The imaging system 40 may detect the human hand 15 by detecting at least one fiduciary marker, such as the passive fiducial marker 42 and/or one or more of the active fiducial markers 44. Further, any of the disclosed sensors (e.g., the extension sensors 12, the pressure sensors 14, the extension zones 22, the compression zones 24, and the like) may detect an impact event associated with the condition of the human hand (block 92). The sensors then may produce a signal indicative of the condition of the human hand at the point in time and within the reference environment (block 93).

The signal indicative of the condition of the human hand may be provided to the computer 50 (block 94). Using the computer 50, the signal of indicative of the condition of the human hand may be analyzed for ergonomic monitoring of the human hand 15 (block 95). Further, the computer may be used to generate ergonomic data associated with the hand 15 (block 96). The ergonomic data associated with the hand 15 may include, but is not limited to including at least one of task related ergonomic impact data, environmental health data, environmental safety data, or ergonomic automation data.

Such ergonomic data is useful in a variety of workplace related research fields for better optimizing production, optimizing workplace efficiency, optimizing placement efficiency within a workplace, providing greater safety capabilities in a work place, providing health monitoring for operators within a workplace, and any other research in a workplace which may be derived from data associated with conditions of hands of operators within a workplace. The apparatus and methods of the present disclosure may provide higher fidelity results for ergonomic data, especially as it pertains to monitoring of the hand.

It will be appreciated that the present disclosure provides apparatus and methods for monitoring conditions of a human hand. While only certain embodiments have been set forth, alternatives and modifications will be apparent from the above description to those skilled in the art. These and other alternatives are considered equivalents and within the spirit and scope of this disclosure and the appended claims.

What is claimed is:

1. An apparatus for monitoring a condition of a human hand, relative to a reference environment, the apparatus comprising:
at least one fiducial marker, the fiducial marker being in the field of view of an imaging system, the imaging system for monitoring the human hand via the fiducial marker in the reference environment at at least one discrete point in time;
at least one sensor capable of detecting an impact event associated with the condition of the human hand, the at least one sensor generating a signal indicative of the condition of the human hand at the at least one discrete point in time and within the reference environment.

2. The apparatus of claim 1, wherein the condition associated with the human hand includes at least one of a hand location, a hand orientation, a joint position of a joint of the human hand, a movement of the human hand, a repetition of hand movement, a pressure on the human hand, or a vibration of the human hand.

3. The apparatus of claim 1, further comprising at least one temperature sensor for detecting a temperature associated with a condition of the human hand, the temperature sensor generating a signal indicative of the condition of the human hand at the at least one discrete point in time and within the reference environment.

4. The apparatus of claim 1, further comprising at least one vibration sensor for detecting a vibration associated with a condition of the human hand, the vibration sensor generating a signal indicative of the condition of the human hand at the at least one discrete point in time and within the reference environment.

5. The apparatus of claim 1, wherein the at least one fiducial marker includes, at least, a passive fiducial marker.

6. The apparatus of claim 1, wherein the passive fiducial marker is a Quick Response (QR) code.

7. The apparatus of claim 1, wherein the at least one fiducial marker includes, at least, an active fiducial marker.

8. The apparatus of claim 7, wherein the active fiducial marker is a light emitting diode (LED) providing light pulses detectable by the imaging system.

9. A method for monitoring a condition of a human hand, relative to a reference environment, the method comprising:
detecting the human hand using an imaging system at at least one discrete point in time, the imaging system detecting the human hand by detecting at least one fiducial marker associated with the human hand;
detecting an impact event associated with the condition of the human hand using at least one sensor; and
generating, using the at least one sensor, a signal indicative of the condition of the human hand at the at least one discrete point in time and within the reference environment.

10. The method of claim 9, further comprising:
providing the signal indicative of the condition of the human hand to a computer;
analyzing, using the computer, the signal indicative of the condition of the human hand for ergonomic monitoring of the human hand; and
generating, using the computer, ergonomic data associated with the human hand.

11. The method of claim 10, wherein the ergonomic data includes at least one of task related ergonomic impact data, environmental health data, environmental safety data, or ergonomic automation data.

12. A glove, the glove being wearable on a human hand and configured for monitoring a condition of the human hand, relative to a reference environment, the glove comprising:
a fabric, the fabric movable with the human hand when the glove is worn by the human hand;
at least one fiducial marker, the fiducial marker being in the field of view of an imaging system, the imaging system for monitoring the human hand via the fiducial marker in the reference environment at at least one discrete point in time;
a plurality of sensors operatively associated with the fabric, the plurality of sensors including at least one impact sensor capable of detecting an impact event associated with the condition of the human hand at the at least one discrete point in time and within the reference environment.

13. The glove of claim 12, wherein the plurality of sensors include fabric-embedded sensors.

14. The glove of claim 13, wherein the fabric-embedded sensors are subdivided into a plurality of fabric compression zones, each of the fabric compression zones capable of providing a signal indicative of a compression event at its respective location on the fabric.

15. The glove of claim 14, wherein each of the plurality of fabric compression zones is located, on the fabric, at a location associated with an interior of a joint of the human hand.

16. The glove of claim 13, wherein the fabric-embedded sensors are subdivided into a plurality of fabric extension zones, each of the fabric extension zones capable of providing a signal indicative of an extension event at its respective location on the fabric.

17. The glove of claim 16, wherein each of the plurality of fabric compression zones is located, on the fabric, at a location associated with an exterior of a joint of the human hand.

18. The glove of claim 13, wherein the fabric-embedded sensors include at least one of a soft textile sensor, a conductive elastic yarn, an elastomeric polymer, or an elastic conductive ribbon.

19. The glove of claim 12, further comprising at least one temperature sensor for detecting a temperature associated with a condition of the human hand, the temperature sensor generating a signal indicative of the condition of the human hand at the at least one discrete point in time and within the reference environment.

20. The glove of claim 12, further comprising a motion sensor for detecting motion associated with a condition of the human hand at the at least one discrete point in time and within the reference environment.
